(19) United States
(12) Patent Application Publication
(10) Pub. No.: US 2024/0370784 A1
(43) Pub. Date: Nov. 7, 2024

(54) METHODS AND SYSTEMS FOR ANALYZING PATIENT LEAKAGE

(71) Applicant: KONINKLIJKE PHILIPS N.V., EINDHOVEN (NL)

(72) Inventors: MARIANA NIKOLOVA-SIMONS, EINDHOVEN (NL); JORN OP DEN BUIJS, EINDHOVEN (NL)

(21) Appl. No.: 18/686,098

(22) PCT Filed: Aug. 31, 2022
(86) PCT No.: PCT/EP2022/074139

§ 371 Date: Feb. 23, 2024

Related U.S. Application Data
(60) Provisional application No. 63/239,652, filed on Sep. 1, 2021.

(51) Int. Cl. G06Q 10/04; G06Q 10/063; G06Q 40/08; G16H 10/60

(52) U.S. Cl. CPC G06Q 10/04; G06Q 10/063; G06Q 40/08; G16H 10/60

(57) ABSTRACT

A method (100) for analyzing and predicting network leakage for a healthcare network using a network leakage analysis system (200), comprising: (i) receiving (120), at the network leakage analysis system, information about one or more patients; (ii) extracting (130), from the received information, one or more patient leakage features about each of the one or more patients; (iii) analyzing (150), by a trained leakage prediction model of the network leakage analysis system, the one or more patient leakage features about each of the one or more patients to generate a leakage prediction for each of the one or more patients; and (iv) providing (160), via a user interface of the network leakage analysis system, a report comprising the leakage prediction for each of the one or more patients.

### FIELD OF THE DISCLOSURE

The present disclosure is directed generally to methods and systems for analyzing, visualizing, and predicting network leakage for a healthcare network using a network leakage analysis and prediction system.

### BACKGROUND

Healthcare organizations (HCO) must navigate both the fee-for-service and the value-based world. This means they need to grow revenues while demonstrating better value to payers and taking on more risk. At the same time, as HCOs manage larger networks of physicians and facilities, they stand to lose considerable revenue and control when patients are referred to other healthcare networks.

Patient leakage denotes the process of patients seeking out-of-network care or being referred out-of-network by healthcare providers. Patient leakage is sometimes known as network leakage or referral leakage. Patient leakage can represent a major impediment to reaching HCO goals in providing high-quality care, coordinating efficient care for patients, and reducing healthcare costs. Even if patients are receiving care outside of their HCO, the latter may still be fully or partially responsible for the associated expenses, including specialty care, primary care services, hospitalizations, and more. Keeping patients within the care of participating providers is the best way to limit expenses caused by leakage.

With patient leakage contributing to significant revenue loss, healthcare executives are prioritizing better referral management. For a majority of healthcare executives (e.g., 87%), patient leakage is a high priority. However, a significant portion of hospitals, medical groups, and physician practices do not track patient leakage. For example, it has been reported that approximately 23% of HCOs do not monitor patient leakage. Even HCOs who do track patient leakage do not fully understand how to reduce the number of patients seeking care beyond their network. Accordingly, approximately 20% of HCOs do not understand where and why patient leakage occurs. Failing to manage patient leakage has a high cost. Over 40% of HCO executives report they are losing 10% or more of annual revenues, 19% are losing over 20% of revenues due to leakage, and 23% don't know how much they are losing.

### SUMMARY OF THE DISCLOSURE

Accordingly, there is a continued need for methods and systems that track, analyze, and predict patient leakage. Various embodiments and implementations herein are directed to a method and system configured to analyze and visualize network leakage for a healthcare network using a network leakage analysis system. The network leakage analysis system receives information about one or more patients, and extracts one or more patient leakage features about each of the one or more patients. A trained leakage prediction model of the network leakage analysis system analyzes the one or more patient leakage features about each of the one or more patients to generate a leakage prediction for each of the one or more patients, and a user interface of the system provides a report comprising the leakage prediction for each of the one or more patients. According to an embodiment, the system analyzes the one or more patient leakage features about each of the one or more patients to determine whether a healthcare treatment or healthcare visit by one or more of the patients was an out-of-network healthcare treatment or healthcare visit, and labels the out-of-network healthcare treatment or healthcare visit as an out-of-network healthcare treatment or healthcare visit. The system then notifies the healthcare network to the labeled out-of-network healthcare treatment or healthcare visit. According to an embodiment, the system generates a patient profile for at least one of the one or more patients based on the received patient information and/or extracted patient leakage features about that patient, and uses the patient profile to generate one or more intervention recommendations for the patient configured to prevent patient leakage of the patient. A report comprising the leakage prediction for each of the one or more patients and the generated one or more intervention recommendations is provided to the healthcare network.

Generally, in one aspect, a method for analyzing network leakage for a healthcare network using a network leakage analysis system is provided. The method includes: (i) receiving, at the network leakage analysis system, information about one or more patients: (ii) extracting, from the received information, one or more patient leakage features about each of the one or more patients: (iii) analyzing, by a trained leakage prediction model of the network leakage analysis system, the one or more patient leakage features about each of the one or more patients to generate a leakage prediction for each of the one or more patients; and (iv) providing, via a user interface of the network leakage analysis system, a report comprising the leakage prediction for each of the one or more patients.

According to an embodiment, the method further includes the step of training the leakage prediction model of the network leakage analysis system.

According to an embodiment, the method further includes the steps of: analyzing the one or more patient leakage features about each of the one or more patients to determine whether a healthcare treatment or healthcare visit by one or more of the patients was an out-of-network healthcare treatment or healthcare visit, wherein the one or more patients were or are in-network patients: labeling the out-of-network healthcare treatment or healthcare visit as an out-of-network healthcare treatment or healthcare visit; and notifying the healthcare network to the labeled out-of-network healthcare treatment or healthcare visit.

According to an embodiment, the method further includes the steps of: generating a report comprising a summary of labeled out-of-network healthcare treatment or healthcare visits; and providing, via the user interface of the network leakage analysis system, the report to the healthcare network.

According to an embodiment, the received patient information and the labeled out-of-network healthcare treatments or healthcare visits are utilized to train the leakage prediction model of the network leakage analysis system.

According to an embodiment, the information about one or more patients is received from one or more of: (i) claims data about the one or more patients; (ii) personal emergency response system data; (iii) electronic medical records data; and (iv) patient home monitoring data.

According to an embodiment, the patient information comprises geographic information, and wherein the report comprises a geographic visualization of the leakage prediction for the one or more patients. According to an embodiment, the geographic visualization comprises patient leakage for one or more geographic regions comprising the healthcare network and/or surrounding the healthcare network.

According to an embodiment, the method further includes the steps of: generating a patient profile for at least one of the one or more patients based on the received patient information and/or extracted patient leakage features about that patient; and generating, based on the patient profile, one or more intervention recommendations for the patient, wherein the intervention recommendation is configured to prevent patient leakage of the patient; wherein the report comprising the leakage prediction for each of the one or more patients further comprises the generated one or more intervention recommendations.

According to an embodiment, the report comprises an alert to a healthcare professional about one or more of the leakage prediction and the one or more intervention recommendations for the patient.

According to a second aspect is a system for analyzing network leakage for a healthcare network. The system includes: a trained leakage prediction model configured to generate a leakage prediction for a patient using one or more patient leakage features about the patient; a processor configured to: (i) receive, at the network leakage analysis system, information about one or more patients; (ii) extract, from the received information, one or more patient leakage features about each of the one or more patients; (iii) analyze, by the trained leakage prediction model, the one or more patient leakage features about each of the one or more patients to generate a leakage prediction for each of the one or more patients; and a user interface configured to provide a report comprising the leakage prediction for each of the one or more patients.

According to an embodiment, the processor is further configured to: analyze the one or more patient leakage features about each of the one or more patients to determine whether a healthcare treatment or healthcare visit by one or more of the patients was an out-of-network healthcare treatment or healthcare visit, wherein the one or more patients were or are in-network patients; label the out-of-network healthcare treatment or healthcare visit as an out-of-network healthcare treatment or healthcare visit; and notify the healthcare network to the labeled out-of-network healthcare treatment or healthcare visit.

According to an embodiment, the processor is further configured to: generate a report comprising a summary of labeled out-of-network healthcare treatment or healthcare visits; and provide, via the user interface of the network leakage analysis system, the report to the healthcare network.

According to an embodiment, the processor is further configured to: generate a patient profile for at least one of the one or more patients based on the received patient information and/or extracted patient leakage features about that patient; and generate, based on the patient profile, one or more intervention recommendations for the patient, wherein the intervention recommendation is configured to prevent patient leakage of the patient: wherein the report comprising the leakage prediction for each of the one or more patients further comprises the generated one or more intervention recommendations.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for analyzing network leakage, in accordance with an embodiment.

FIG. 2 is a schematic representation of a network leakage analysis system, in accordance with an embodiment.

FIG. 3 is a flowchart of a method for training a leakage prediction model, in accordance with an embodiment.

FIG. 4 is a schematic representation of a network leakage analysis system using electronic health records (EHR) and personal emergency response system (PERS) data, in accordance with an embodiment.

FIG. 5 is a schematic representation of a network leakage analysis system using claims data, in accordance with an embodiment.

FIG. 6 is a schematic representation of a network leakage analysis system, in accordance with an embodiment.

FIG. 7 is a flowchart of a method for analyzing patient leakage using claims data, in accordance with an embodiment.

FIG. 8 is a schematic representation of generating leakage prediction using claims data, in accordance with an embodiment.

FIG. 9 is a flowchart of a method for generating leakage prediction using claims data, in accordance with an embodiment.

FIG. 10 is a schematic representation of generating leakage prediction using EHR and PERS data, in accordance with an embodiment.

FIG. 11 is a flowchart of a method for generating leakage prediction using EHR and PERS data, in accordance with an embodiment.

FIG. 12 is a flowchart of a method for analyzing patient leakage using EHR and PERS data, in accordance with an embodiment.

FIG. 13 is a visualization of identified patient leakage and/or predicted patient leakage, in accordance with an embodiment.

FIG. 14 is a visualization of identified patient leakage and/or predicted patient leakage, in accordance with an embodiment.

FIG. 15 is a visualization of identified patient leakage and/or predicted patient leakage, in accordance with an embodiment.

FIG. 16 is a flowchart of a method for generating intervention recommendations, in accordance with an embodiment.

FIG. 17 is a schematic representation of a patient profile, in accordance with an embodiment.

### DETAILED DESCRIPTION OF EMBODIMENTS

The present disclosure describes various embodiments of a system and method configured to track, analyze, and predict patient leakage for a healthcare network. More generally, Applicant has recognized and appreciated that it would be beneficial to provide a method and system to predict and prevent patient leakage. Accordingly, a trained network leakage analysis system tracks, predicts, and reports patient leakage for a healthcare network, optionally with leakage prevention recommendations, which can be leveraged to mitigate or prevent future patient leakage. The system receives information about one or more patients, and extracts one or more patient leakage features about each of the one or more patients. A trained leakage prediction model of the network leakage analysis system analyzes the one or more patient leakage features about each of the one or more patients to generate a leakage prediction for each of the one or more patients, and a user interface of the system provides a report comprising the leakage prediction for each of the one or more patients. According to an embodiment, the system analyzes the one or more patient leakage features about each of the one or more patients to determine whether a healthcare treatment or healthcare visit by one or more of the patients was an out-of-network healthcare treatment or healthcare visit, and labels the out-of-network healthcare treatment or healthcare visit as an out-of-network healthcare treatment or healthcare visit. The system then notifies the healthcare network to the labeled out-of-network healthcare treatment or healthcare visit. According to an embodiment, the system generates a patient profile for at least one of the one or more patients based on the received patient information and/or extracted patient leakage features about that patient, and uses the patient profile to generate one or more intervention recommendations for the patient configured to prevent patient leakage of the patient. A report comprising the leakage prediction for each of the one or more patients and the generated one or more intervention recommendations is provided to the healthcare network.

According to an embodiment, the network leakage analysis system tracks patient leakage retrospectively, identifies potential patient leakage prospectively, and integrates patient leakage with other data-driven insights. Indeed, there is a continued need to integrate patient leakage predictions with other data-driven insights in order to generate tailored interventions. Accordingly, the system discloses a Patient Leakage Analyzer (PLA), which leverages either claims data or a combination of PERS and EHR data. The PLA assesses whether a patient's clinical event such as inpatient or outpatient encounter is a leakage, i.e., whether the clinical event was with a clinical provider out-of-network of HCO system. If yes, the clinical event is flagged as leakage and the HCO is notified. Second, the system comprises a Patient Leakage Predictor (PLP) that uses the PLA output to annotate existing clinical events as leakage/no-leakage and then trains a novel predictive model identifying patients at high risk of leakage. Third, the system comprises an Integration Engine (IE) which combines the patient's risk of leakage with other risk models to create patient profiles. Each of these profiles triggers a time-dependent bundle of tailored interventions and notifications to the care team members that will provide these interventions.

According to an embodiment, the systems and methods described or otherwise envisioned herein can, in some non-limiting embodiments, be implemented as an element for a commercial product for patient analysis or monitoring, such as Philips® Connected Care Solutions (available from Koninklijke Philips NV, the Netherlands), or any suitable patient or care facility system.

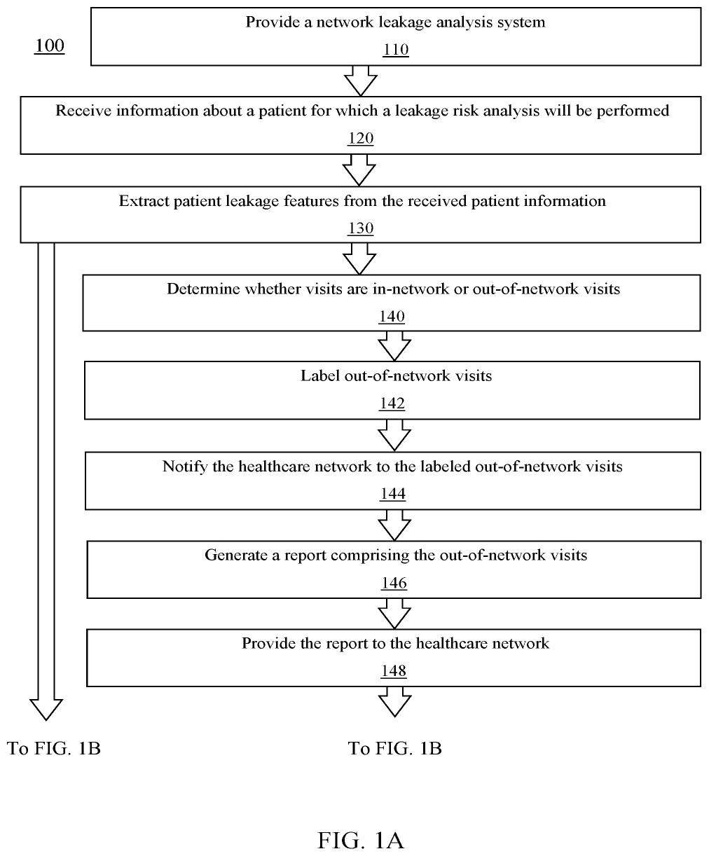

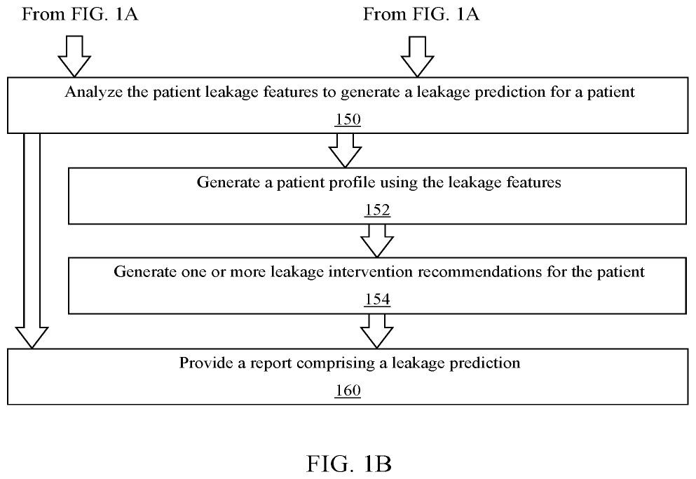

Referring to FIG. 1, in one embodiment is a flowchart of a method 100 for analyzing network leakage for a healthcare network using a network leakage analysis system. The methods described in connection with the figures are provided as examples only, and shall be understood not to limit the scope of the disclosure. The network leakage analysis system can be any of the systems described or otherwise envisioned herein. The network leakage analysis system can be a single system or multiple different systems.

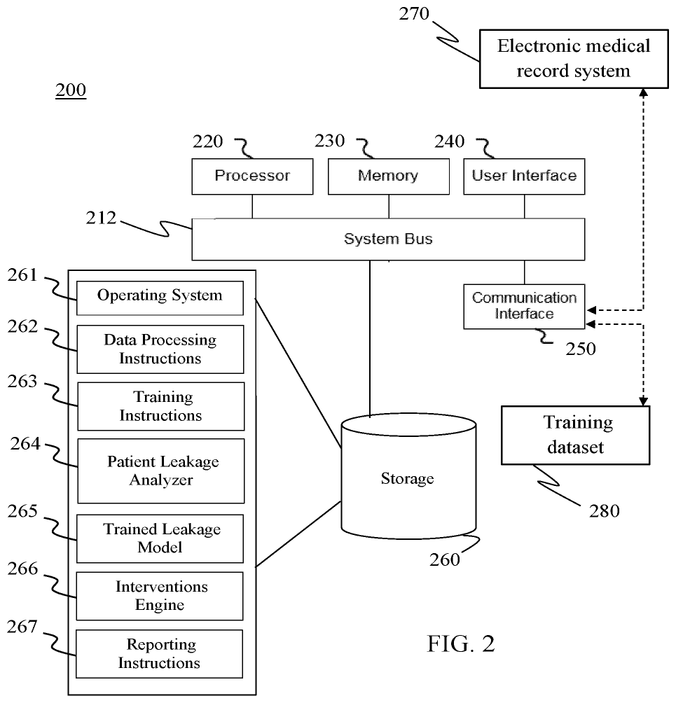

At step 110 of the method, a network leakage analysis system is provided. Referring to an embodiment of a network leakage analysis system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, network leakage analysis system 200 can be any of the systems described or otherwise envisioned herein. Other elements and components of network leakage analysis system 200 are disclosed and/or envisioned elsewhere herein.

At step 120 of the method, the network leakage analysis system receives information about a patient for which an analysis will be performed. According to an embodiment, the information comprises a plurality of features about the patient. Only some, or all, of the information about the patient and features may ultimately be utilized by the network leakage analysis system. The information about the patient and plurality of features may comprise, for example, demographic and/or medical features about the patient. For example, demographic information or features may comprise age, gender, past healthcare facility visits or admissions, and other demographic information. Medical information or features may comprise vital sign information about the patient, including but not limited to physiologic vital signs such as heart rate, blood pressure, respiratory rate, apnea, SpO₂, invasive arterial pressure, noninvasive blood pressure, physiological measurements other than vital data such as physical observations, patient diagnosis or medication condition such as cardiac disease, psychological disorders, chronic obstructive pulmonary disease, and more, among many other types of medical information. Many other types of patient information are possible. Accordingly, the received information can be any information relevant to a patient leakage analysis.

The network leakage analysis system can receive patient information from a variety of different sources, including any source that comprises one or more patient features. According to an embodiment, the network leakage analysis system is in communication with an electronic medical records database from which the patient information and one or more of the plurality of features may be obtained or received. The electronic medical records database may be a local or remote database and is in communication with the network leakage analysis system 200. According to an embodiment, the network leakage analysis system comprises an electronic medical record database or system 270 which is optionally in direct and/or indirect communication with system 200. According to another embodiment, the network leakage analysis system may obtain or receive the plurality of features from equipment or a healthcare professional obtaining that information directly from the patient. According to another embodiment, the network leakage analysis system may obtain or receive the plurality of features from home monitoring equipment, such as a wearable device, PERS, CPAP machine, or any other device utilized by a patient outside of a hospital or other direct healthcare facility. According to an embodiment, the network leakage analysis system may query an electronic medical record database or system, comprising fast healthcare interoperability resources (FHIR) for example, to obtain the patient information.

The patient information received by the network leakage analysis system may be processed by the system according to methods for data handling and processing/preparation, including but not limited to the methods described or otherwise envisioned herein. The patient information received by the network leakage analysis system may be utilized, before or after processing, immediately or may be stored in local or remote storage for use in further steps of the method.

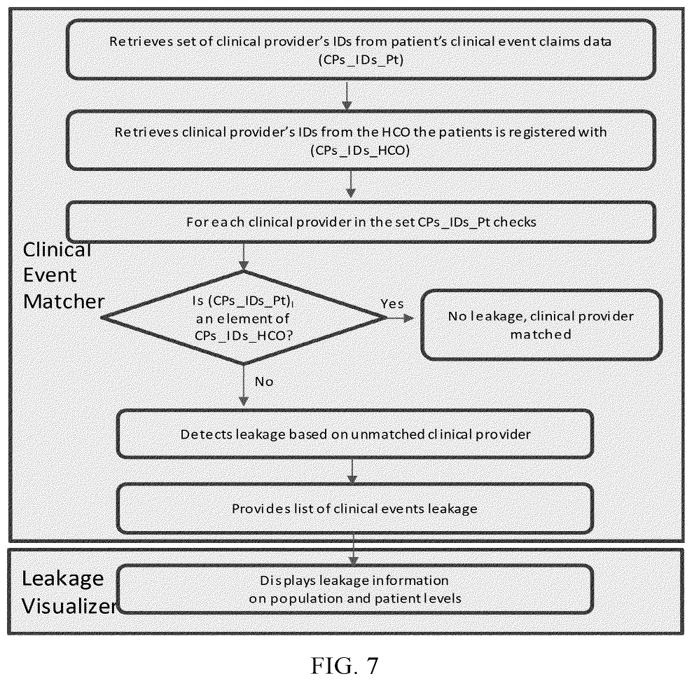

According to an embodiment, the patient information comprises claims data which is leveraged by the system to assess whether a patient's clinical event like inpatient or outpatient encounter is a leakage, such as whether the event was with a clinical provider out-of-network of the HCO system. For example, the system can comprise a module or component such as a Clinical Event Matcher (see FIGS. 6 and 7) that retrieves all clinical provider's IDs (CPs_IDs_Pt) from patient claims data. The Clinical Event Matcher also retrieves a set of clinical provider's IDs (CPs_IDs_HCO) from the HCO the patient is registered with. Third, for each clinical provider extracted from the claims data, the Clinical Event Matcher checks whether (CPs_IDs_Pt)₁ is an element of CPs_IDs_HCO. If no matching is found in the clinical provider's ID the clinical events associated with (CPs_IDs_Pt)₁ are flagged as out-of-network (leakage) and their data are stored in a leakage database.

Claims data may not be available for leakage analysis, or other data may be desired. According to an embodiment, the system may merge different data sources to detect a specific leakage. For example, the system may detect emergency room (ER) leakage by merging a healthcare provider EHR with PERS data, among other data sources.

The probability of patient leakage is especially high in the case of emergency care. For example, Emergency Medical Services (EMS/Ambulance) policy is to bring patients to the nearest ER best equipped to treat the emergency at hand. Accordingly, the network leakage analysis system can be configured to track or otherwise address patient ER leakage which occurs when a patient is transported to out-of-network ER of hospitals belonging to a particular HCO.

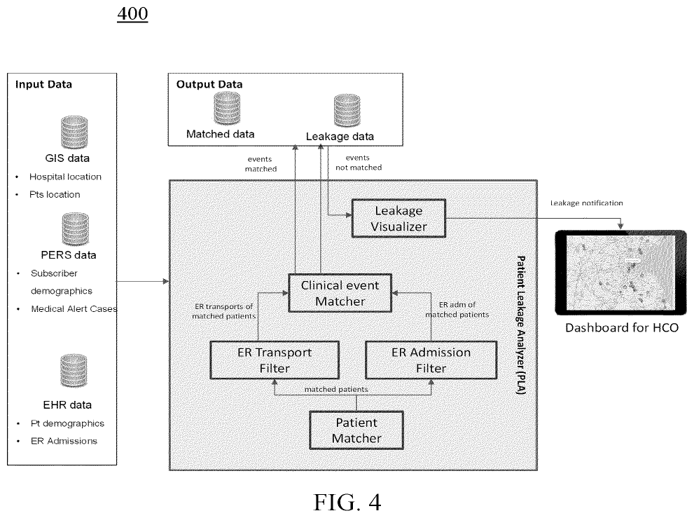

Referring to FIG. 4, in one embodiment, is a representation of a Patient Leakage Analyzer (PLA) system that utilizes EHR and PERS data. According to an embodiment, a PERS facilitates EMS transport of elderly people living independently at home to a hospital ER. The PERS can consist of three components: (i) a personal help button that is worn either as a necklace or a bracelet 24 hours a day; (ii) an in-home communication system; and (iii) an emergency call center. PERS subscribers may press the help button at any time to activate the in-home communication system that connects them to a call center. In case a subscriber is unable to press the help button, e.g. due to unconsciousness, and uses the AutoAlert version of PERS, an automatic call for help will be placed to the call center whenever a fall is detected. The call center associate assesses the situation and contacts either an informal responder (e.g. a neighbor, a family member) or an EMS based on patient's specific situation, and follow up to confirm that help has arrived.

Notably, in addition to PERS, other remote monitoring can also be used to detect ER visits outside of the hospital network. For example, biotelemetry obtained through a device such as a wearable or a smartwatch can detect out-of-range heart rhythms outside of the hospital. In case of detection of a heart rhythm that requires emergency medical response, the location of the patient could be tracked via the smartwatch or phone to determine to which ER the patient is transported.

Each interaction of a PERS subscriber with a call center associate is recorded as a case, which has a number of attributes, e.g. case type, case situation and case outcome, such as the examples in Table 1. A case type indicates either regular tests of PERS, maintenance or servicing of the system, accidental button presses, or adverse events like incidents. The case situation describes why a subscriber pressed the personal help button. In case of an incident the case outcome indicates: (i) what type of assistance the PERS subscriber gets such as emergency-, responder-, or self-assistance; and (ii) whether the subscriber was transported to an ER. The case outcome contains the information whether a subscriber is transported to a hospital ER.

TABLE 1
Examples of Case Attributes

| | Case Situation for | |
|---|---|---|
| Case Type | Type = Incident | Case Outcome for Type = Incident |
| Welcome | Bleeding/Injury | Emergency Assist - Transportation |
| Test | Anxious | Emergency Assist - No |
| | | Transportation |
| Maintenance | Breathing Problem | Emergency Assist - No Status |
| Check-In | Dizziness | Responder Assist - Transportation |
| Accidental | Request Responder | Responder Assist - No |
| | | Transportation |
| Incident | Stroke | Sub Assisted Self |

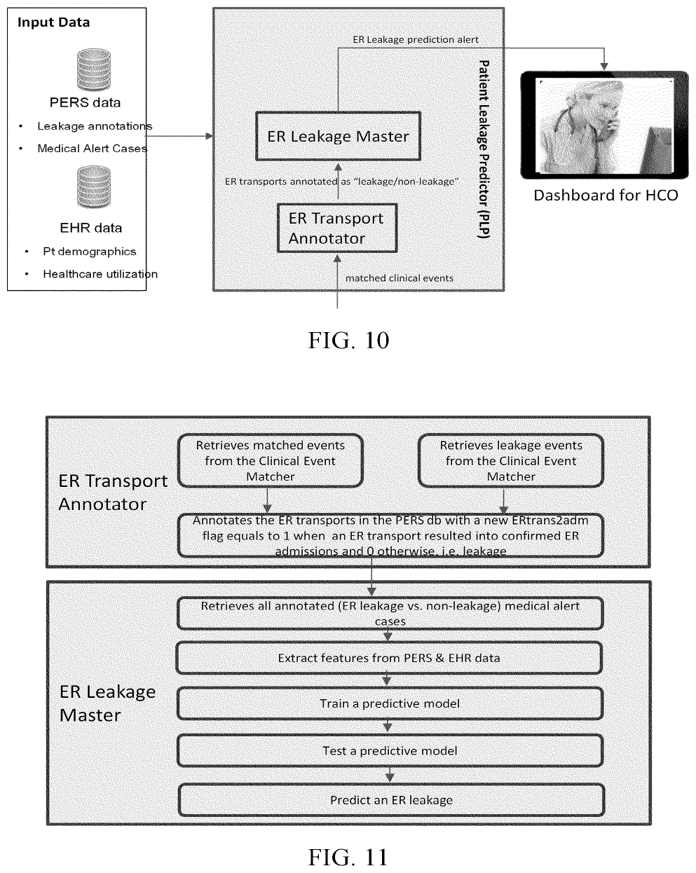

According to an embodiment, the system comprises a Patient Leakage Analyzer (PLA) which leverages PERS and EHR data to assess whether a PERS subscriber with an ER transport had an emergency admission to in-network hospital system. If no, then the ER transport is flagged as leakage and the HCO is notified. Second, this embodiment describes the PLP that uses the PLA output to annotate existing ER transports as leakage/no-leakage and trains a novel predictive model identifying patients at high risk of ER leakage (FIG. 10). Notably, according to this embodiment, the PLA comprises a Patient Matcher and ER Transport and ER Admission Filters in addition to the Clinical Event Matcher and the Leakage Visualizer (FIG. 4).

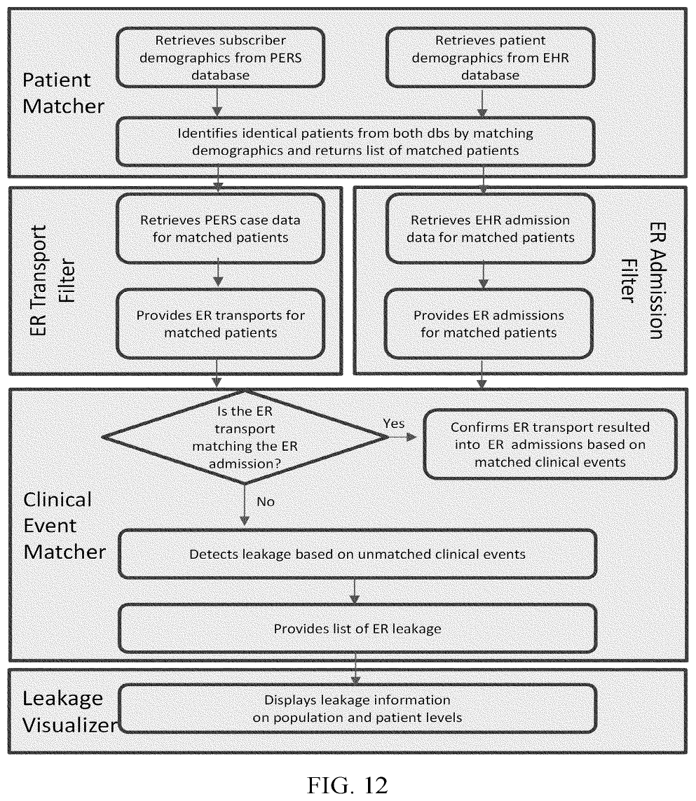

According to an embodiment, the Patient Leakage Analyzer comprises a Patient Matcher that can receive as input one or more of: (i) a subscriber's demographics such as from a PERS database of a Medical Alert Service provider; and (ii) a patient's demographics from an EHR database of a HCO. The Patient Matcher uses these data to identify identical patients, i.e. patients that are present in both databases. The output of the Patient Matcher is a data set of demographics for the matched patients (FIG. 12).

According to an embodiment, the Patient Leakage Analyzer comprises an ER Transport Filter that retrieves medical alert cases from the PERS database as well as gets the demographics information of the matched patients from the Patient Matcher. From the case data it filters out the cases belonging to the matched patients with case outcome indicating an ER transport (FIG. 12).

According to an embodiment, the Patient Leakage Analyzer comprises an ER Admission Filter that retrieves admission data (i.e., in-patient and out-patient encounters) from the EHR database as well as gets the demographics information of the matched patients from the Patient Matcher. From the admission data it filters out the ER admissions belonging to the matched patients (FIG. 12).

According to an embodiment, the Patient Leakage Analyzer comprises a Clinical Event Matcher that receives the data for ER transports and ER admissions from the ER Transport Filter and ER Admission Filter, respectively. Based on these data it determines which ER transports resulted in-network ER admissions (i.e. within the HCO hospital system). If no match found the ER transports are flagged as out-of-network (leakage) and their data are stored in a leakage database (FIG. 12).

According to an embodiment, the network leakage analysis system stores matched data and/or leakage data in local or remote storage. The matched data and/or leakage data may be stored or may be utilized in another step of the method.

According to an embodiment, the information about one or more patients is received from one or more of: (i) claims data about the one or more patients; (ii) PERS data; (iii) EHR data; and (iv) patient home monitoring data. According to an embodiment, the patient information comprises geographic information as described or otherwise envisioned herein.

At step 130 of the method, the network leakage analysis system extracts patient leakage features from the patient information received in step 120 of the method. The features can be any of the features described or otherwise envisioned herein. The system can be trained to identify and extract the patient features from the received patient information, using any of a wide variety of algorithms, methods, or systems for identifying and extracting patient data. The plurality of identified and extracted patient features may be utilized immediately or may be stored in local or remote storage for use in further steps of the method. According to an embodiment, the patient information and/or patient features may undergo data processing at any stage.

At optional step 140 of the method, the network leakage analysis system analyzes the one or more patient leakage features about each of the one or more patients to determine whether a healthcare treatment or healthcare visit by one or more of the patients was an out-of-network healthcare treatment or healthcare visit. According to an embodiment, some or all of the patients are or previously were in-network patients.

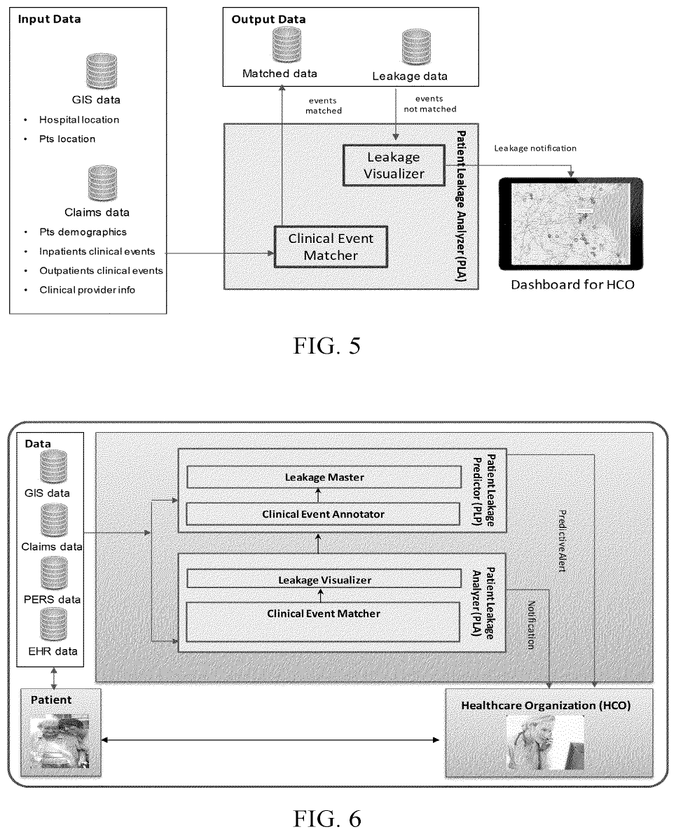

Referring to FIG. 5, in one embodiment, is a schematic representation of a network leakage analysis system comprising a Patient Leakage Analyzer (PLA) configured to determine whether a healthcare treatment or healthcare visit by one or more of the patients was an out-of-network healthcare treatment or healthcare visit, based on claims data. Referring to FIG. 6, in one embodiment, is a schematic representation of a network leakage analysis system also comprising a Patient Leakage Analyzer (PLA) in conjunction with a Patient Leakage Predictor (PLP) as described or otherwise envisioned herein. Referring to FIG. 7, in one embodiment, is a flowchart of a method for analyzing patient leakage, using claims data. Referring to FIG. 9, in one embodiment, is a flowchart of a method for generating a leakage prediction for a patient using a variety of claims data as input.

According to an embodiment, the network leakage analysis system is configured to determine whether a healthcare treatment or healthcare visit by one or more of the patients was an out-of-network healthcare treatment or healthcare visit. For example, the system can comprise a module, component, or algorithm configured to analyze out-of-network treatment of in-network patients. According to an embodiment, the patient information comprises claims data which is leveraged by the system to assess whether a patient's clinical event like inpatient or outpatient encounter is a leakage, such as whether the event was with a clinical provider out-of-network of the HCO system.

At optional step 142 of the method, the network leakage analysis system labels healthcare treatment or healthcare visit identified in step 140 as being out-of-network, as an out-of-network healthcare treatment or healthcare visit. This can be accomplished using any method or system for labeling events or data or patient information.

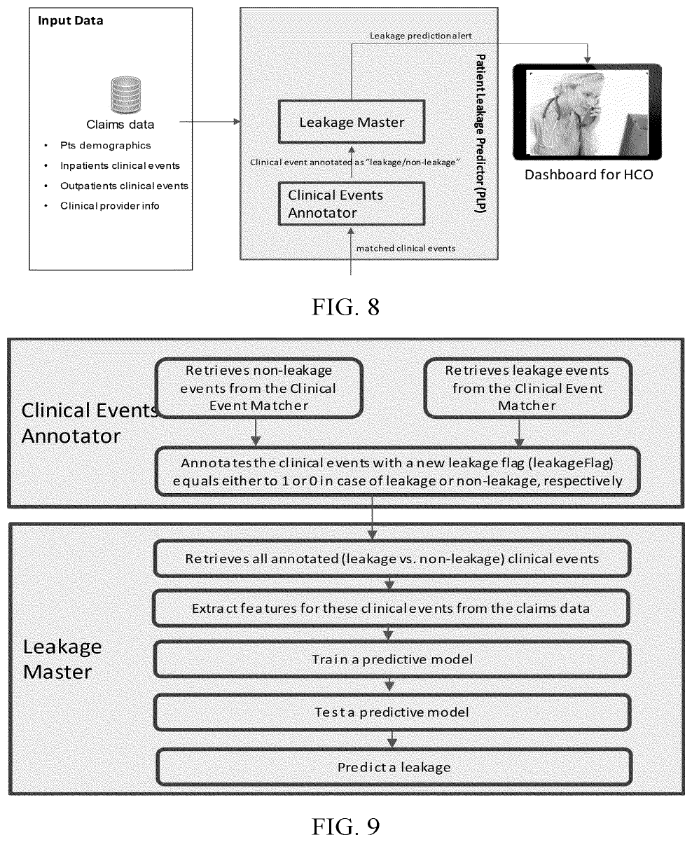

According to an embodiment, the network leakage analysis system can comprise a Clinical Event Annotator (FIG. 8) or other module, component, or algorithm that labels out-of-network healthcare treatments or healthcare visits. The Clinical Event Annotator can first retrieve data on both matched as well as unmatched medical events from the Clinical Event Matcher (or other module, component, or algorithm that identifies visits as in-network or out-of-network). The Clinical Event Annotator can also annotate clinical events with a new leakage flag (such as, for example, leakageFlag) which equals either to 1 or 0 in case of leakage or non-leakage, respectively. This output of the Clinical Event Annotator can be used by the Leakage Master or other module, component, or algorithm of the network leakage analysis system.

At optional step 144 of the method, the network leakage analysis system notifies the healthcare network to the labeled out-of-network healthcare treatment or healthcare visit. For example, the system may comprise a reporting module or system, and/or an alerting module or system, configured to generate and/or provide a report and/or an alert to the healthcare network, such as to a user of the healthcare network. Accordingly, optional step 146 of the method, the network leakage analysis system generates a report comprising a summary of labeled out-of-network healthcare treatment or healthcare visits, or other summary of patient leakage identified by the system, and may also comprise information about one or more individual patients identified as patient leakage by the system.

At optional step 148 of the method, the network leakage analysis system provides the report to the healthcare network via a user interface of the network leakage analysis system. The user interface can be any mechanism for communicating the information. The information may be communicated by wired and/or wireless communication to any device. For example, the system may communicate the information to a mobile phone, computer, laptop, wearable device, and/or any other device configured to allow display and/or other communication of the information.

According to an embodiment, the network leakage analysis system can comprise a Leakage Visualizer that is configured to receive input from the system, such as from a leakage database and/or external data sources such as geographical information systems (GIS) to generate data for visualizations of patient leakage on both levels—an individual patient or the HCO patient population. Other aspects of the report or alert provided via a user interface to a user of the network leakage analysis system are described or otherwise envisioned herein.

At step 150 of the method, a trained leakage prediction model of the network leakage analysis system analyzes the one or more patient leakage features about each of the one or more patients to generate a leakage prediction for each of the one or more patients. The trained leakage prediction model can generate a leakage prediction using a wide variety of different classifier and/or machine learning algorithms as described or otherwise envisioned herein. In addition to the one or more patient leakage features about each of the one or more patients, the trained leakage prediction model of the network leakage analysis system can use a wide variety of other data and data sources as input for the model.

Referring to FIG. 8, in one embodiment, is a schematic representation of generating a leakage prediction for a patient using claims data as input. Referring to FIG. 9, in one embodiment, is a flowchart of a method for generating a leakage prediction for a patient using claims data as input. Referring to FIG. 10, in one embodiment, is a flowchart of a method for generating a leakage prediction for a patient using EHR and PERS data. Referring to FIG. 11, in one embodiment, is a flowchart of a method for generating a leakage prediction for a patient using EHR and PERS data. Referring to FIG. 12, in one embodiment, is a flowchart of a method for analyzing patient leakage using a variety of data as input.

According to an embodiment, the network leakage analysis system can comprise a Leakage Master (FIG. 8) configured to retrieve all annotated (leakage vs. non-leakage) clinical events. The Leakage Master can extract features from the claims data to train and test a new predictive model which calculates a probability of upcoming clinical event to result in a leakage for a given HCO. Finally, the Leakage Master can apply the predictive model to each patient of a given HCO to calculate a risk of upcoming clinical event leakage. If the risk is high a leakage prediction alert is sent to the HCO. The Leakage Master can support healthcare providers to proactively intervene and potentially prevent patient leakage.

According to an embodiment, the network leakage analysis system can annotate clinical events as leakage or no-leakage and can then train a novel predictive model identifying patients at high risk of leakage. The system may comprise, therefore, the Clinical Event Annotator and Leakage Master (FIG. 8) as described or otherwise envisioned herein. According to an embodiment, the network leakage analysis system can comprise an ER Transport Annotator and ER Leakage Master (FIG. 10), or equivalent modules, components, or algorithms, as described or otherwise envisioned herein. According to an embodiment, the ER Transport Annotator retrieves data on both matched as well as unmatched medical events from the Clinical Event Matcher. The ER Transport Annotator can annotate the ER transports in the PERS data base with a new ERtrans2adm flag equals to 1 (or similar flag or label) when an ER transport resulted into confirmed ER admissions and 0 otherwise. Thus, the ER leakage includes al ER transports with ERtrans2adm flag=0 (or similar flag or label). According to an embodiment, the system comprises an ER Leakage Master that retrieves all annotated (ER leakage vs. non-leakage) medical alert cases. Second, the ER Leakage Master extracts patient's features form PERS and EHR data to train and test a new predictive model which calculates a probability of upcoming ER transport to result in a leakage for a given HCO. Finally, the ER Leakage Master can apply the predictive model to each patient of a given HCO to calculate a risk of upcoming ER leakage. If the risk is high an ER leakage prediction alert can be sent to the HCO.

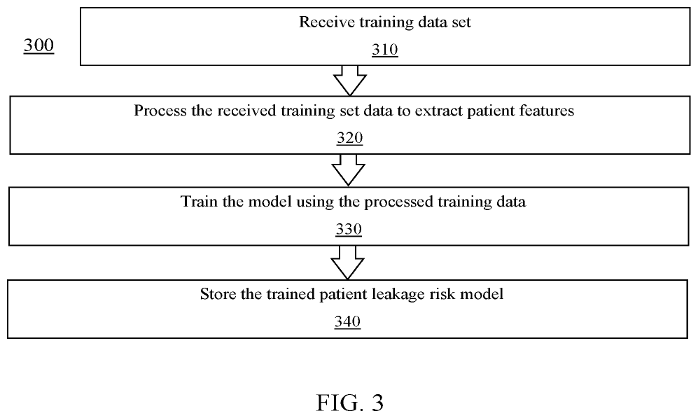

Referring to FIG. 3, in one embodiment, is a method 300 for training the leakage prediction model of the network leakage analysis system. At step 310 of the method, the system receives a training data set comprising training data about a plurality of patients. The training data can comprise demographic and/or medical features about the patient. For example, demographic information or features may comprise age, gender, past healthcare facility visits or admissions, and other demographic information. Medical information or features may comprise vital sign information about the patient, including but not limited to physiologic vital signs such as heart rate, blood pressure, respiratory rate, apnea, SpO₂, invasive arterial pressure, noninvasive blood pressure, physiological measurements other than vital data such as physical observations, patient diagnosis or medication condition such as cardiac disease, psychological disorders, chronic obstructive pulmonary disease, and more, among many other types of medical information. Many other types of patient information are possible. Accordingly, the received information can be any information relevant to a patient leakage analysis. According to an embodiment, the training data may comprise retrospective admission/discharge/transfer (ADT) data. The training data may be stored in and/or received from one or more databases. The database may be a local and/or remote database. For example, the system may comprise a database of training data, such as database 280 in FIG. 2.

According to an embodiment, the network leakage analysis system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 320 of the method, the system processes the received information to extract leakage prediction features about one or more of the plurality of patients. The leakage prediction features may be any features which will be utilized to train the leakage risk model, such as any leakage prediction features that can or will be utilized by the trained algorithm for leakage risk analysis for a future patient. Feature extraction can be accomplished by a variety of embodiments for feature identification, extraction, and/or processing, including any method for extracting features from a dataset. The outcome of a feature processing step or module of the leakage risk analysis system is a set of leakage prediction features about a plurality of patients, which thus comprises a training data set that can be utilized to train the classifier.

At step 330 of the method, the system trains the machine learning algorithm, which will be the algorithm utilized in analyzing patient information as described or otherwise envisioned. The machine learning algorithm is trained using the extracted features according to known methods for training a machine learning algorithm. According to an embodiment, the algorithm is trained, using the processed training dataset, to generate a leakage analyze for a patient. According to an embodiment, the algorithm is also trained, using the processed training dataset, to generate one or more intervention recommendations. At step 340 of the method, the trained model is stored for future use. According to an embodiment, the model may be stored in local or remote storage.

At step 160 of the method in FIGS. 1A and 1B, a report comprising the leakage prediction for each of the one or more patients—and potentially other information—is provided via a user interface of the network leakage analysis system. Alternatively, the information may be communicated by wired and/or wireless communication to another device. For example, the system may communicate the information to a mobile phone, computer, laptop, wearable device, and/or any other device configured to allow display and/or other communication of the information.

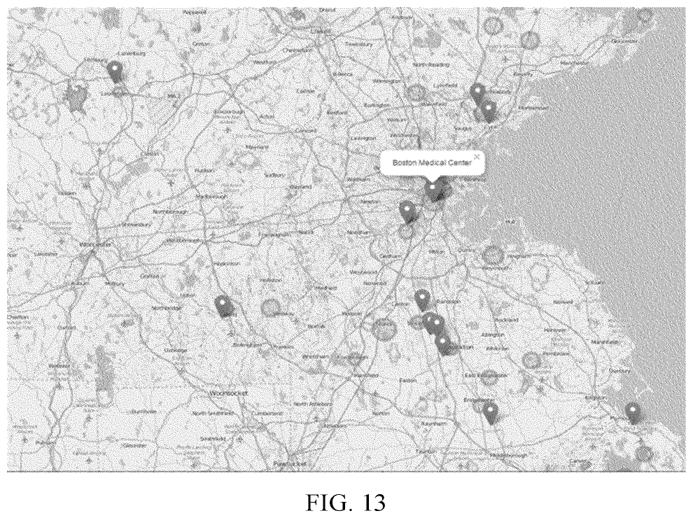

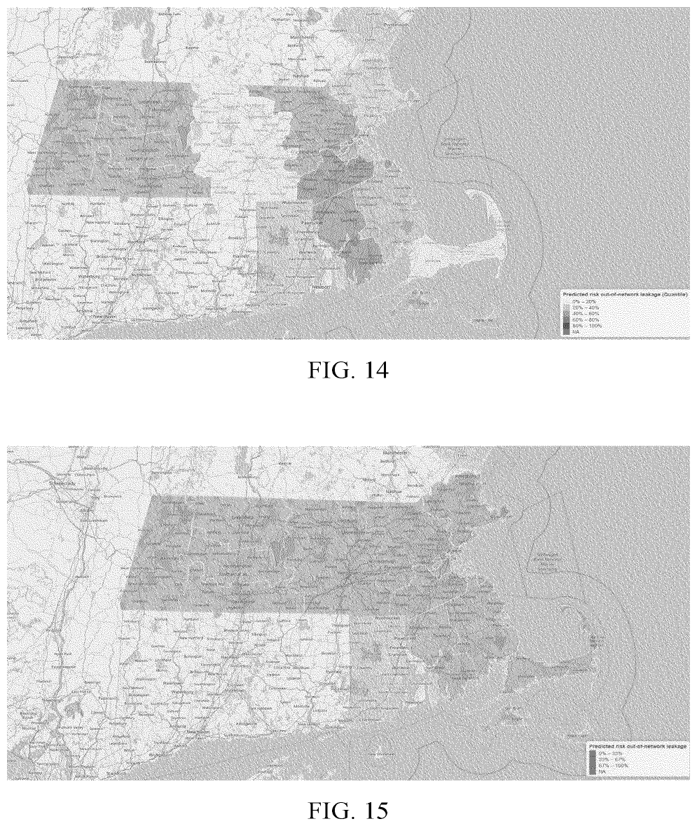

Referring to FIGS. 13-15 are reports or visualizations of identified patient leakage and/or predicted patient leakage, in accordance with certain embodiments. According to an embodiment, the network leakage analysis system may comprise a Leakage Visualizer (FIG. 6) or similar module, component, or algorithm configured to generate a visualization of identified patient leakage and/or predicted patient leakage.

In one embodiment, the existing patient leakage is visualized by zip code on a map, together with nearby out-of-network hospitals. In another embodiment, the predicted patient's leakage is visualized, i.e. a predictive model of patient leakage is used to predict the probability of leakage on patient level, which is then aggregated on a population (e.g., county or zip code) level. The visualization shows per county the average predicted leakage. Color coding/grading can depict different quantiles of risk. Counties with higher risk of leakage can be colored in darker red, other coloring schemes include green/yellow/red. The figures show there is a range of neighboring counties with high predicted leakage risk. This provides novel insights to the network, which they can leverage such as when attracting new patients or expanding their hospital locations.

In FIG. 13, the visualization comprises patient leakage together with nearby out-of-network hospitals. Circles indicate areas where leakage occurred, and the size of the circles represent the degree of leakage. Out-of-network hospitals are indicated by markers.

Returning to method 100 in FIGS. 1A and 1B, at optional step 152 of the method the system generates a patient profile for one or more patients based on the received patient information and/or extracted patient leakage features about that patient. At optional step 154 of the method, the system generates, based on the generated patient profile, one or more intervention recommendations for the patient, with the intervention recommendation being configured to prevent patient leakage of the patient. Accordingly, a report provided by the system can comprise identified leakage, predicted leakage, and/or one or more generated intervention recommendations for one or more patients.

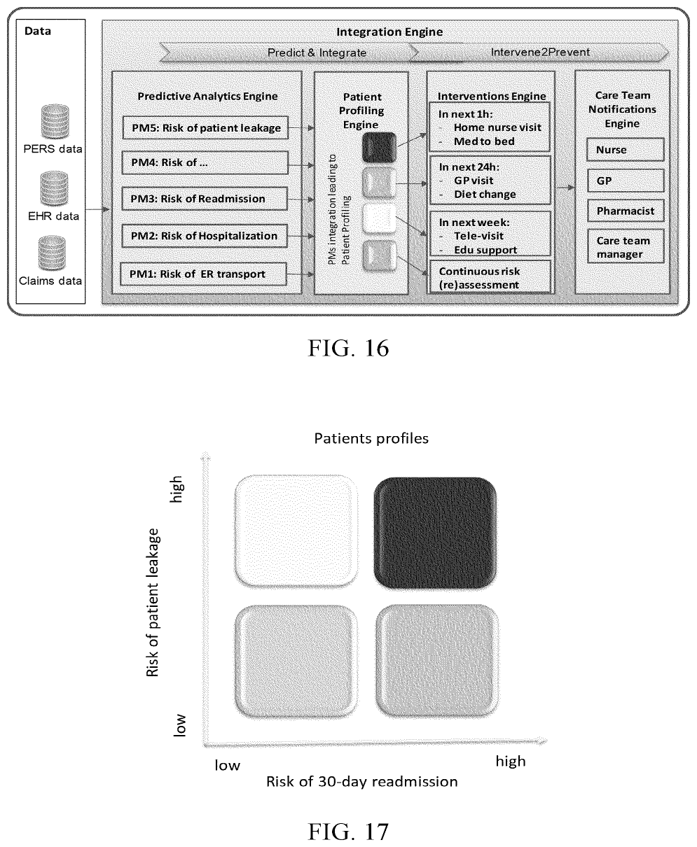

Referring to FIG. 16, in one embodiment, is a flowchart of a method for generating intervention recommendations for one or more patients. Referring to FIG. 17, in one embodiment, is a schematic representation of a patient profile based on risk scores.

According to an embodiment, the network leakage analysis system comprises an Integration Engine (FIG. 16) or similar module, component, or algorithm that is configured to combine a patient leakage prediction with results of one or more other predictive models to trigger patients tailored interventions.

According to an embodiment, the network leakage analysis system comprises a Predictive Analytics Engine or similar module, component, or algorithm configured to run all the predictive models (PM) including the PM on patient leakage. The outputs of the models, i.e. the risk scores, are fed into the Patient Profiling Engine (FIG. 16).

According to an embodiment, the network leakage analysis system comprises a Patient Profiling Engine (FIG. 16) or similar module, component, or algorithm configured to generate patient profiles based on (selection of the) PM risk scores. For example, combining the risk of patient leakage with the risk of 30-day readmission could lead to the patient profiles illustrated in FIG. 17, among many other options.

According to an embodiment, the network leakage analysis system comprises an Interventions Engine (FIG. 16) or similar module, component, or algorithm. Using the generated patient profile, the Intervention Engine identifies care gaps present and creates bundle of interventions. A time stamp can be assigned to each bundle of interventions to indicate a priority of how quickly the care team members must act. Specifically, for patient at high risk of leakage the bundle of intervention can be derived from a patient pathway that excludes out-of-network healthcare services. Many other options are possible.

According to an embodiment, the network leakage analysis system comprises a Care Team Notification Engine (FIG. 16) or similar module, component, or algorithm configured to create notifications and checklists for the members of the care team (such as a Nurse Practitioner, Home Nurse, GP, Pharmacist, Hospitalist, Care team manager, etc.). Many other options are possible.

Referring to FIG. 2 is a schematic representation of a network leakage analysis system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, the system may comprise, among other instructions or data, an electronic medical record system 270, training dataset 280, data processing instructions 262, training instructions 263, Patient Leakage Analyzer (PLA) 264, trained leakage prediction model or Patient Leakage Predictor (PLP) 265, Interventions Engine 266, and/or reporting instructions 267.

According to an embodiment, the electronic medical record system 270 is an electronic medical records database from which the information about the patient, including the plurality of leakage prediction features, may be obtained or received. The electronic medical records database may be a local or remote database and is in communication the network leakage analysis system 200. According to an embodiment, the patient network leakage analysis system comprises an electronic medical record database or system 270 which is optionally in direct and/or indirect communication with system 200.

According to an embodiment, the training data set 280 is a dataset that may be stored in a local or remote database and is in communication with the network leakage analysis system 200. The training data can comprise medical information about a patient, including but not limited to demographics, physiological measurements such as vital data, physical observations, and diagnosis, as well as claims data, EHR data, PERS data, and/or EMS data, among many other types of medical information.

According to an embodiment, data processing instructions 262 direct the system to retrieve and process input data which is used to train the leakage prediction model by the Patient Leakage Predictor (PLP) 264. The input data can be used by the system as needed, such as from electronic medical record system 270 among many other possible sources. As described above, the input data can comprise a wide variety of input types from a wide variety of sources, including home monitoring devices and other patient monitoring devices, among other devices and systems.

According to an embodiment, the data processing instructions 262 also direct the system to process the input data to generate a plurality of leakage prediction features related to medical information for a plurality of patients, which are used to train the classifier. This can be accomplished by a variety of embodiments for feature identification, extraction, and/or processing. The outcome of the feature processing is a set of leakage prediction features related to leakage analysis for a patient, which thus comprises a training data set that can be utilized to train the model 265.

According to an embodiment, training instructions 263 direct the system to utilize the processed data to train the leakage prediction model 265. The leakage prediction model can be any machine learning algorithm, classifier, or model sufficient to utilize the type of input data provided, and to generate a patient leakage risk/prediction. Thus, the system comprises a trained leakage prediction model 265 configured to generate a leakage risk/prediction for a patient, as described or otherwise envisioned herein.

According to an embodiment, the Patient Leakage Analyzer (PLA) 264 directs the system to process input data and track or otherwise analyze patient leakage. According to an embodiment, the PLA assesses whether a patient's clinical event such as inpatient or outpatient encounter is a leakage, i.e., whether the clinical event was with a clinical provider out-of-network of HCO system. The PLA can then flag, label, or otherwise identify an event and/or patient with an out-of-network label.

According to an embodiment, the Interventions Engine or Integration Engine 266 directs the system to generate a patient profile for a patient using received patient information and/or extracted patient leakage features about that patient, and directs the system to generate, based on the patient profile, one or more intervention recommendations for the patient designed or configured to prevent patient leakage. According to an embodiment, the Interventions Engine combines a patient's risk of leakage with other risk models to create patient profiles. Each of these profiles triggers a time-dependent bundle of tailored interventions and notifications to the care team members that will provide these interventions. According to an embodiment, the Intervention Engine identifies care gaps present and creates bundle of interventions. A time stamp can be assigned to each bundle of interventions to indicate a priority of how quickly the care team members must act. Specifically, for patient at high risk of leakage the bundle of intervention can be derived from a patient pathway that excludes out-of-network healthcare services. Many other options are possible.

According to an embodiment reporting instructions 267 direct the network leakage analysis system to generate and provide to a user via a user interface information comprising a generated report comprising the leakage prediction for each of the one or more patients, leakage analysis for one or more patients, and/or one or more generated intervention recommendations. The information may be communicated by wired and/or wireless communication to another device. For example, the system may communicate the information to a mobile phone, computer, laptop, wearable device, and/or any other device configured to allow display and/or other communication of the information.

According to an embodiment, the network leakage analysis system is configured to process many thousands or millions of datapoints in the input data used to train the classifier, as well as to process and analyze the received plurality of patient features. For example, generating a functional and skilled trained classifier using an automated process such as feature identification and extraction and subsequent training requires processing of millions of datapoints from input data and the generated features. This can require millions or billions of calculations to generate a novel trained classifier from those millions of datapoints and millions or billions of calculations. As a result, each trained classifier is novel and distinct based on the input data and parameters of the machine learning algorithm, and thus improves the functioning of the network leakage analysis system. Thus, generating a functional and skilled trained classifier comprises a process with a volume of calculation and analysis that a human brain cannot accomplish in a lifetime, or multiple lifetimes.

In addition, the network leakage analysis system can be configured to continually receive patient features, perform the analysis, and provide periodic or continual updates via the report provided to a user for the patient. This requires the analysis of thousands or millions of datapoints on a continual basis to optimize the reporting, requiring a volume of calculation and analysis that a human brain cannot accomplish in a lifetime. By providing an improved patient leakage analysis or prediction, this novel network leakage analysis system has an enormous positive effect on preventing patient leakage compared to prior art systems.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “cither or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of.” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for analyzing and predicting network leakage for a healthcare network using a network leakage analysis system, comprising:
receiving, at the network leakage analysis system, information about one or more patients;
extracting, from the received information, one or more patient leakage features about each of the one or more patients;
analyzing, by a trained leakage prediction model of the network leakage analysis system, the one or more patient leakage features about each of the one or more patients to generate a leakage prediction for each of the one or more patients; and
providing, via a user interface of the network leakage analysis system, a report comprising the leakage prediction for each of the one or more patients.

2. The method of claim 1, further comprising the step of training the leakage prediction model of the network leakage analysis system.

3. The method of claim 1, further comprising the steps of:
analyzing the one or more patient leakage features about each of the one or more patients to determine whether a healthcare treatment or healthcare visit by one or more of the patients was an out-of-network healthcare treatment or healthcare visit, wherein the one or more patients were or are in-network patients;
labeling the out-of-network healthcare treatment or healthcare visit as an out-of-network healthcare treatment or healthcare visit;
notifying the healthcare network to the labeled out-of-network healthcare treatment or healthcare visit.

4. The method of claim 3, further comprising the steps of:
generating a report comprising a summary of labeled out-of-network healthcare treatment or healthcare visits; and
providing, via the user interface of the network leakage analysis system, the report to the healthcare network.

5. The method of claim 3, wherein the received patient information and the labeled out-of-network healthcare treatments or healthcare visits are utilized to train the leakage prediction model of the network leakage analysis system.

6. The method of claim 1, wherein the information about one or more patients is received from one or more of: (i) claims data about the one or more patients; (ii) personal emergency response system data; (iii) electronic medical records data; and (iv) patient home monitoring data.

7. The method of claim 1, wherein the patient information comprises geographic information, and wherein the report comprises a geographic visualization of the leakage prediction for the one or more patients.

8. The method of claim 7, wherein the geographic visualization comprises patient leakage for one or more geographic regions comprising the healthcare network and/or surrounding the healthcare network.

9. The method of claim 1, further comprising the steps of:
generating a patient profile for at least one of the one or more patients based on the received patient information and/or extracted patient leakage features about that patient; and
generating, based on the patient profile, one or more intervention recommendations for the patient, wherein the intervention recommendation is configured to prevent patient leakage of the patient;
wherein the report comprising the leakage prediction for each of the one or more patients further comprises the generated one or more intervention recommendations.

10. The method of claim 8, wherein the report comprises an alert to a healthcare professional about one or more of the leakage prediction and the one or more intervention recommendations for the patient.

11. A system for analyzing and predicting network leakage for a healthcare network, comprising:
a trained leakage prediction model configured to generate a leakage prediction for a patient using one or more patient leakage features about the patient;
a processor configured to: (i) receive, at the network leakage analysis system, information about one or more patients; (ii) extract, from the received information, one or more patient leakage features about each of the one or more patients; (iii) analyze, by the trained leakage prediction model, the one or more patient leakage features about each of the one or more patients to generate a leakage prediction for each of the one or more patients; and
a user interface configured to provide a report comprising the leakage prediction for each of the one or more patients.

12. The system of claim 11, wherein the processor is further configured to:
analyze the one or more patient leakage features about each of the one or more patients to determine whether a healthcare treatment or healthcare visit by one or more of the patients was an out-of-network healthcare treatment or healthcare visit, wherein the one or more patients were or are in-network patients; label the out-of-network healthcare treatment or healthcare visit as an out-of-network healthcare treatment or healthcare visit; and notify the healthcare network to the labeled out-of-network healthcare treatment or healthcare visit.

13. The system of claim 12, wherein the processor is further configured to: generate a report comprising a summary of labeled out-of-network healthcare treatment or healthcare visits; and provide, via the user interface of the network leakage analysis system, the report to the healthcare network.

14. The system of claim 11, wherein the processor is further configured to: generate a patient profile for at least one of the one or more patients based on the received patient information and/or extracted patient leakage features about that patient; and generate, based on the patient profile, one or more intervention recommendations for the patient, wherein the intervention recommendation is configured to prevent patient leakage of the patient; wherein the report comprising the leakage prediction for each of the one or more patients further comprises the generated one or more intervention recommendations.

15. The system of claim 11, wherein the report comprises an alert to a healthcare professional about one or more of the leakage prediction and the one or more intervention recommendations for the patient.

16. A non-transitory computer readable medium storing instructions for analyzing and predicting network leakage for a healthcare network using a network leakage analysis system that, when executed by one or more processors, cause the one or more processors to:
receive, at the network leakage analysis system, information about one or more patients;
extract, from the received information, one or more patient leakage features about each of the one or more patients;
analyze, by a trained leakage prediction model of the network leakage analysis system, the one or more patient leakage features about each of the one or more patients to generate a leakage prediction for each of the one or more patients; and
provide, via a user interface of the network leakage analysis system, a report comprising the leakage prediction for each of the one or more patients.

17. The non-transitory computer readable medium of claim 16, wherein the instructions further cause the one or more processors to:
analyze the one or more patient leakage features about each of the one or more patients to determine whether a healthcare treatment or healthcare visit by one or more of the patients was an out-of-network healthcare treatment or healthcare visit, wherein the one or more patients were or are in-network patients;
label the out-of-network healthcare treatment or healthcare visit as an out-of-network healthcare treatment or healthcare visit;
notify the healthcare network to the labeled out-of-network healthcare treatment or healthcare visit.

18. The non-transitory computer readable medium of claim 17, wherein the received patient information and the labeled out-of-network healthcare treatments or healthcare visits are utilized to train the leakage prediction model of the network leakage analysis system.

19. The non-transitory computer readable medium of claim 16, wherein the instructions further cause the one or more processors to:
generate a report comprising a summary of labeled out-of-network healthcare treatment or healthcare visits; and
provide, via the user interface of the network leakage analysis system, the report to the healthcare network.

20. The non-transitory computer readable medium of claim 16, wherein the instructions further cause the one or more processors to:
generate a patient profile for at least one of the one or more patients based on the received patient information and/or extracted patient leakage features about that patient; and
generate, based on the patient profile, one or more intervention recommendations for the patient, wherein the intervention recommendation is configured to prevent patient leakage of the patient;
wherein the report comprising the leakage prediction for each of the one or more patients further comprises the generated one or more intervention recommendations.
